(19) United States
(12) Patent Application Publication
(10) Pub. No.: US 2025/0372238 A1
(43) Pub. Date: Dec. 4, 2025

(54) METHOD FOR CARRYING OUT 3D SEGMENTATION OF A SAMPLE

(71) Applicant: COMMISSARIAT A L'ÉNERGIE ATOMIQUE ET AUX ENERGIES ALTERNATIVES, Paris (FR)

(72) Inventors: Guillaume GODEFROY, Grenoble Cedex 09 (FR); Chiara PAVIOLO, Grenoble Cedex 09 (FR)

(73) Assignee: COMMISSARIAT A L'ÉNERGIE ATOMIQUE ET AUX ENERGIES ALTERNATIVES, Paris (FR)

(21) Appl. No.: 19/223,182

(22) Filed: May 30, 2025

(30) Foreign Application Priority Data
Jun. 1, 2024 (FR) FR2405742

(51) Int. Cl. G16H 30/40; G06T 7/10; G06V 10/762; G16H 30/20

(52) U.S. Cl. CPC G16H 30/40; G06T 7/10; G06V 10/762; G16H 30/20

(57) ABSTRACT

Segmenting method, for carrying out 3D segmentation of a sample, the sample comprising at least one biological object, the sample developing over time, such that at least one biological object divides or changes shape or position over time, the method comprising:
at various times, acquiring a stack of images (P(t)) of the sample;
segmenting images, such as to obtain masks corresponding to each biological object;
implementing a segmentation algorithm that is said to be prompted, such as to use masks obtained, for an object, in an image, to define masks, for the same object, in another image.

### TECHNICAL FIELD

The technical field of the invention is observation of microscopic 3D structures, for example cells or embryos during development.

### PRIOR ART

In the field of biology, it may be useful to study the development of cellular objects, such as embryos. In the early stages of development, the number of cells in an embryo gradually multiplies, forming a morula. Morula is the name given to an embryo when it has at least 16 cells. Before or when it has become a morula, it may be advantageous to monitor the development of an embryo, for the purposes of understanding the mechanisms governing fertilization, initial cell divisions and cellular differentiation. It may also help to understand the causes of developmental abnormalities, and to optimize in vitro fertilization techniques.

Acquisition systems have been developed that allow biological samples to be observed in 3D. Such a system is for example described in EP4207077.

However, the configuration of a multicellular object, such as an embryo, changes over time, and tools allowing cells and how they change over time to be better seen and analysed are required. The invention described below meets this need.

### SUMMARY OF THE INVENTION

A first subject of the invention is a segmenting method, for carrying out 3D segmentation of a sample, the sample comprising at least one biological object, the sample developing over time, such that at least one biological object divides or changes shape or position over time, the method comprising:
a) at various times, acquiring a stack of images of the sample, each image of the stack of images showing one sectional plane of the sample, each stack of images showing the sample at a given time, the times defining time slots, such that during each time slot the sample contains the same number of biological objects, at least one stack of images being acquired in each time slot;
b) segmenting at least one image of a stack of images such as to define masks in said image, each mask being bounded by a closed outline;
c) selecting masks defined in step b) depending on predefined selection criteria, such that each selected mask corresponds to one biological object, each selected mask being associated with a position corresponding to a position of the mask in the segmented image;
d) reiterating steps b) and c) on another image of a stack of images of the same time slot, until at least one selected mask is obtained for each biological object of the sample;
e) selecting a stack of images, acquired during a time slot, referred to as the time slot of interest;
f) for at least one biological object, and using at least one mask, selected in the time slot of interest and corresponding to the biological object, carrying out segmentation of at least one image, and preferably of each image of the stack of images selected in step e), such as to determine, in a plurality of images of said stack of images, masks corresponding to the biological object, the segmentation being guided by the position of the selected mask;
g) repeating steps e) to f) for various stacks of images in at least one or in each time slot.

Steps b) to g) are implemented by a processing unit.

Steps e) and f) may be repeated for each stack of images of each time slot.

Step f) may be repeated for each biological object in the sample, in at least one time slot.

The method may comprise, prior to step b), obtaining a number N(Δt) of biological objects contained in the sample, during the or each time slot, N(Δt) being an integer greater than or equal to 1.

The method may comprise, prior to step b), determining the number of biological objects, in at least one time slot, from at least one stack of images acquired during the time slot.

According to one possibility:
the acquisition times are distributed between various time slots, with each time slot corresponding to a number of biological objects in the sample;
the method comprises, prior to step b), clustering each stack of images, such as to assign each stack of images to one of said time slots.

According to one possibility, the method comprises, prior to step b):
i) applying a dimension-reduction algorithm to each stack of images, such as to assign a coordinate to each stack of images in a latent space;
ii) in the latent space, assigning each coordinate to a class;
iii) determining the number of biological objects in the sample, for each stack of images, depending on the class corresponding to the stack of images.

Step i) may comprise using an encoder neural network to obtain, from each stack of images, a code corresponding to each stack of images, the dimension-reduction algorithm being applied to the code.

Step i) may comprise forming an image representative of each stack of images, to be fed to the encoder neural network.

The representative image may be a maximum projection image established for each stack of images.

The respective sectional planes of each image of a given stack of images may be parallel to one another, or, alternatively, angularly inclined with respect to one another.

In step c), at least one selection criterion may be chosen from:
a morphological criterion of the mask;
a maximum overlap between two selected masks.

The morphological criterion may be chosen from:
a minimum area and/or a maximum area bounded by the outline of each mask;
a shape criterion of the outline of each mask.

The shape criterion may correspond to a circular shape or to an ellipsoidal shape of eccentricity below a threshold value.

Step c) may comprise determining a physical property of the sample in a portion of the image corresponding to each mask, the selection criterion depending on the value of said physical property.

The physical property may be an optical property of the sample, for example a refractive index, or a phase shift of light induced by the sample, or an absorbance of light by the sample.

According to one possibility:
in step a), two successively acquired stacks of images are offset by one temporal increment, two adjacent images of a given stack of images being offset by one spatial increment;
each image in which at least one mask has been selected in a step c) is a reference image for the biological object corresponding to the mask;
step f) comprises:
   f-i) selecting a biological object;
   f-ii) selecting an image in which no mask has been defined for the selected biological object, the selected image having a spatial coordinate and a temporal coordinate;
   f-iii) for the biological object selected in substep f-i), selecting a mask corresponding to the selected biological object in a neighbouring reference image neighbouring the image selected in substep f-ii), the neighbouring reference image having respective spatial and temporal coordinates offset by a respective number of spatial and temporal increments less than a predetermined threshold;
   f-iv) transferring the position of the mask selected in substep f-iii) to the image selected in substep f-ii);
   f-v) carrying out segmentation of the image selected in substep f-ii), using the position transferred in substep f-iv), such as to define a mask for the biological object selected in substep f-i), the segmentation being guided by said position of the mask.

Following step f-v), the image selected in substep f-ii) may become a reference image for the biological object selected in substep f-i).

According to one possibility:
in substep f-iii), the position of the selected mask is defined by a bounding box framing said mask;
substep f-iv) comprises transferring the bounding box to the image selected in substep f-ii).

Substeps f-i) to f-v) may be carried out such as to obtain one mask for each biological object of the sample in each image of each stack of images.

The sample may be a multicellular organism, each biological object being one cell.

The sample may be an embryo, each biological object being one cell.

A second subject of the invention is a device for observing a sample, comprising:
an acquisition system, configured to form a stack of images of the sample at various times, each stack of images showing the sample at a given time, the times defining time slots, during which the sample contains the same number of biological objects, such that at least one stack of images corresponds to each time slot;
a processing unit, configured to implement steps b) to g) of a method according to the first subject of the invention, based on the respective stacks of images formed at the various times.

A third subject of the invention is a medium that is connectable to a computer and that contains instructions for implementing steps b) to g) of a method according to the first subject of the invention based on stacks of images of a sample.

The invention will be better understood on reading the description of the examples of embodiment presented, in the remainder of the description, with reference to the figures listed below.

### FIGURES

FIG. 1A schematically shows one example of a device according to the invention.

FIG. 1B schematically shows another example of a device according to the invention.

FIG. 2 illustrates a stack of images.

FIG. 3A illustrates the main steps of a method for carrying out 3D segmentation of a sample according to the invention.

FIG. 3B details substeps of a step of obtaining as many masks as there are cells in a stack of images.

FIG. 3C details substeps of a step of segmenting the images forming a stack of images.

FIG. 4 schematically shows one implementation of an auto-encoder.

FIGS. 5A and 5D show stacks of images at various respective times.

FIGS. 5B and 5E show maximum projection images obtained from the stacks of images shown in FIGS. 5A and 5D, respectively.

FIGS. 5C and 5F schematically show codes obtained by applying an auto-encoder to the maximum projection images shown in FIGS. 5B and 5E, respectively.

FIG. 6 illustrates a projection of stacks of images into a latent space of dimension 2, and distribution of each stack of images into clusters. In FIG. 6, each stack of images has been represented by one point.

FIG. 7 illustrates a variation as a function of time in the class associated with each stack of images.

FIGS. 8A, 8D and 8G show images of a given stack of images.

FIGS. 8B, 8E and 8H show some of the segmentation masks obtained from images 8A, 8D and 8G, respectively.

FIGS. 8C, 8F and 8I show masks selected, following the respective segmentations of images 8A, 8D and 8G.

FIG. 9A shows an image of a stack of images.

FIG. 9B represents segmentation masks obtained by segmenting the image shown in FIG. 9A.

FIGS. 9C, 9D and 9E show reference images, in which a cell has been segmented. FIGS. 9C, 9D and 9E correspond to images temporally and/or spatially offset with respect to the image of FIG. 9A.

FIGS. 9F, 9G and 9H show masks, corresponding to the same object, defined in FIGS. 9C, 9D and 9E respectively.

FIGS. 9I, 9J and 9K show cell masks obtained from the image of FIG. 9A, based on the masks shown in FIGS. 9F, 9G, 9H, respectively.

FIG. 10 shows an application of the 3D segmentation of a sample, for viewing the various cells from which the sample is composed.

### DESCRIPTION OF PARTICULAR EMBODIMENTS

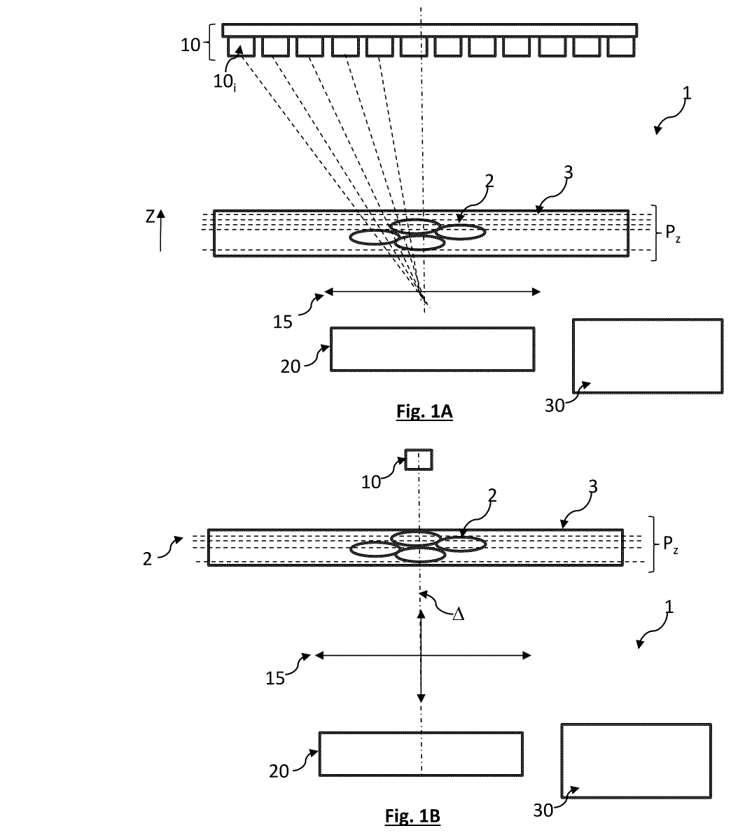

FIG. 1A shows one example of a device 1 allowing the invention to be implemented. The device comprises a light source 11 configured to illuminate a sample 2. The light source is formed from a plurality of elementary sources 11ᵢ, the latter being light-emitting diodes. The sample 2 is a biological sample, the development of which it is desired to observe. Thus, the sample comprises one or more biological objects. The sample develops over time, such that the number and/or position and/or shape of biological objects may change over time. The biological objects may notably divide. By way of non-limiting example, the sample is a cellular sample, such as an embryo, notably a non-human embryo. In this example the embryo is a mouse embryo. The sample is contained in a container 3. The embryo comprises cells, the number and shape of which change over time.

The device comprises a lens 15 coupled to an image sensor 20. During implementation of the device, each elementary light source 11ᵢ is turned on sequentially, and an image of the sample is acquired each time an elementary light source is turned on. This allows illumination of the sample at a variable angle of incidence, one image of the sample being acquired for each angle of incidence. On the basis of the various images acquired, a processing unit 30 makes it possible to reconstruct sectional planes of the sample, which sectional planes are preferably parallel to one another and orthogonal to a Z-axis. The processing unit 30 comprises at least one microprocessor, configured to execute stored instructions and to allow implementation of algorithms. The processing unit 30 implements a tomographic reconstruction algorithm. Patent application EP4207077 describes a device such as shown in FIG. 1A and implementation of an algorithm allowing absorption or phase images to be obtained in sectional planes. In this example, phase images of the sample are used.

FIG. 1B shows another device allowing the invention to be implemented. The device comprises a light source 11 configured to illuminate a sample 2. As in FIG. 1A, the light source may be a light-emitting diode.

The device comprises an image sensor 20 coupled to a lens 15, the latter making it possible to conjugate an object plane, called the focal plane, with the image sensor. The image sensor and the optical system are aligned along an optical axis Δ. The assembly formed by the image sensor and the optical system is configured such that the focal plane may be translated parallel to the optical axis Δ, to various depths inside the sample.

Alternatively, the image sensor is associated with a confocal diaphragm, the latter allowing successive observation of various slices of the sample.

Thus, generally, an acquisition system 1 is provided, this acquisition system being configured to form images of various sectional planes of a sample, so as to form a stack of images. The acquired images, forming the stack of images, may be standard images, absorbance images or phase images or diffraction images or indeed fluorescence images of the sample, when the latter contains a fluorescent marker, or more generally any type of quantity measurable by a microscope.

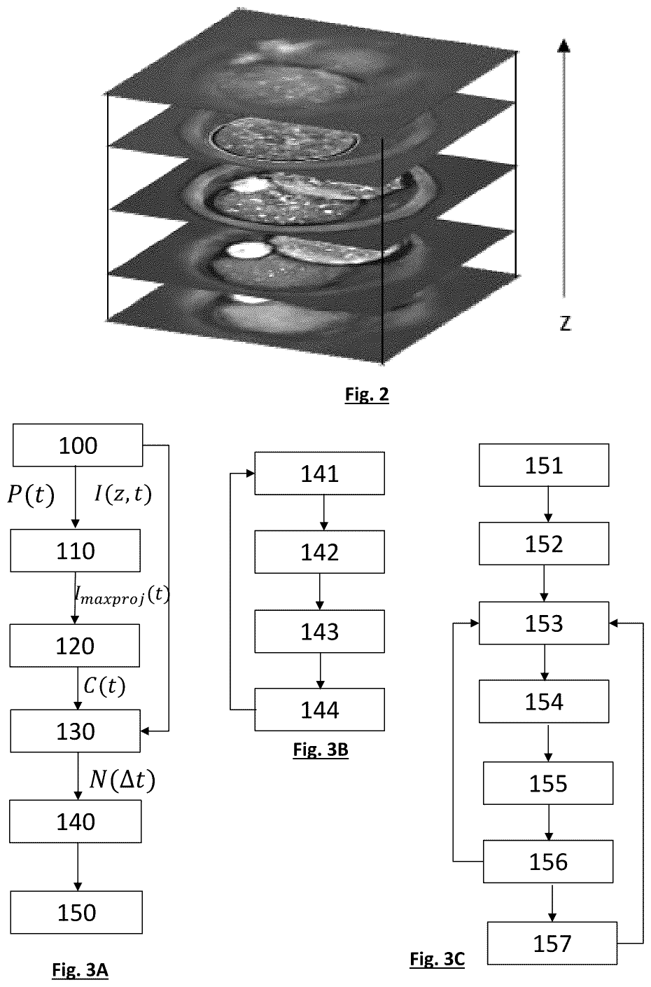

FIG. 2 shows one example of sectional planes of a mouse embryo, obtained with a device such as schematically shown in FIG. 1A.

One objective of the invention is to allow the development of the sample to be followed over time, in three dimensions. According to a complementary aspect, the invention makes it possible to classify phases of development of the sample, based on stacks of images acquired at various times.

FIG. 3A schematically shows various steps implemented by the processing unit 30, or by any other suitable means.

### Step 100: Acquisition of Stacks of Images at Various Times

In this step, a plurality of stacks of images P(t) are formed, at various respective times t. Each stack of images is formed from images acquired, by the image sensor, at each given time. As described with reference to FIGS. 1A and 1B, at each given time, the image sensor acquires a series of images, which are used to form a stack of images. The images of the series of images are considered to have been acquired at the same time, the time difference between the acquisitions of the images of a given series of images being negligible.

Each image of the stack of images corresponds either to an image acquired by the image sensor, or to an image obtained by processing, and notably by tomographic reconstruction, of images of the sample. Each stack of images P(t) contains images I(z, t). The index z corresponds to a spatial index of each image, along the Z-axis, with z₀≤z≤zₘₐₓ. The index t is a temporal index, relating to the acquisition times. The times lie between an initial time t₀ and a final time t_{f}. The period between t₀ and t_{f} is the acquisition period T. The various stacks of images allow information about the spatio-temporal development of the sample to be obtained.

The acquisition period T comprises one or more time slots Δt. During each time slot, the sample is considered to contain the same number of biological objects N(Δt), in the present case the same number of cells. The time slots, and the number of biological objects per time slot, may be known, for example in principle or based on measurements taken using another measuring method. Alternatively, and optionally, the time slots and the number of objects per time slot may be determined from the stack of images, as described in connection with steps 110 to 130.

For clarification purposes, the term “series of images” refers to images acquired by the sensor, from which images a stack of images is obtained, the stack corresponding to images of the sample in various planes, and preferably in various planes parallel to one another. At each time, one series of images is acquired, from which one stack of images is obtained. The number of sectional planes may be from a few tens to a few thousand. In the example shown, between 50 and 150 sectional planes have been taken into account.

According to one possibility, the sectional planes are angularly spaced apart from one another.

Each stack of images depends on the state of a biological sample at a time t. Steps 110 to 130, described below, allow the state of the cellular sample to be followed over time, via dimension reduction (steps 110 to 120) and clustering (step 130).

### Step 110: Compression

During the acquisition period, the cellular sample develops. Based on the stack of images P(t), a clustering algorithm is implemented to identify various phases of the development of the sample: it is a question of identifying the time slots.

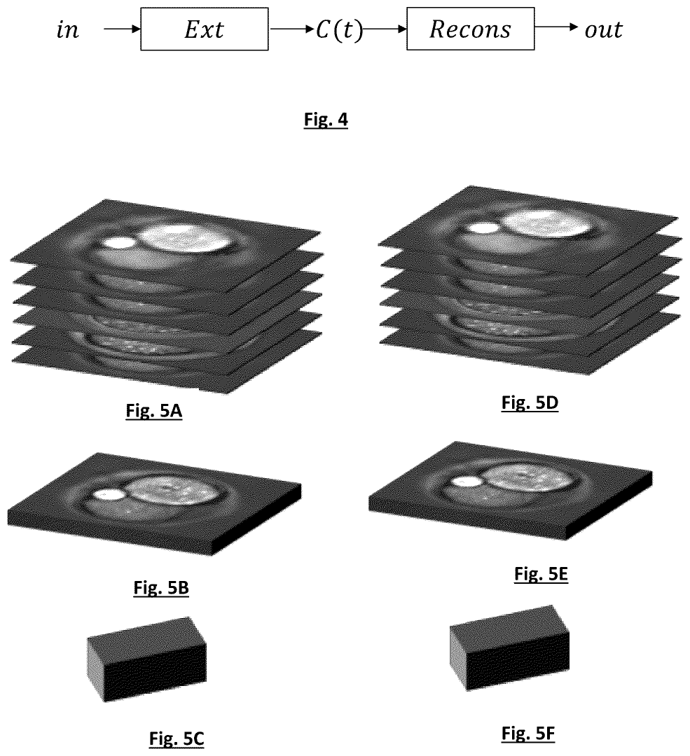

To do this, a code C(t) of each stack of images P(t) is used, which code is of reduced dimension with respect to each stack of images, and contains information concerning it. A code of a stack of images may be obtained by implementing a convolutional neural network of encoder type. It may, for example, be the encoder of an auto-encoder as schematically shown in FIG. 4. In a manner known to those skilled in the art, a neural network of auto-encoder type is a structure comprising an extraction block Ext, called an encoder, which makes it possible to extract relevant information from an input datum In, which is generally of large dimension, defined in an input space. The information extracted by the extraction block is called a code C(t).

The auto-encoder comprises a reconstruction block Recons allowing the code to be reconstructed, such as to obtain an output datum out defined in a space that is generally identical to the input space. The auto-encoder is trained such as to minimize an error between the input datum in and the output datum out. Following training, the code extracted by the extraction block is considered to be representative of the main features of the input datum. In other words, the extraction block allows compression of the information contained in the input datum.

According to one possibility, the output of the auto-encoder is not the image provided as input, but a mask representative of the objects shown in the input image.

The input datum may be a stack of images. However, it is preferable to form an input datum containing compressed information about the stack of images. The input datum of the encoder may be an image, called the maximum projection image Iₘₐₓₚᵣₒⱼ(t). Each image I(z, t) is defined by pixels r. In each pixel r, the maximum projection image is such that

$${I}_{maxproj} ( t , r ) = {max}_{{z}_{0} \leq z \leq {z}_{max}} I ( z , t , r ) \qquad ( 1 )$$

At each given time t, from a stack of images P(t), it is possible to obtain a maximum projection image Iₘₐₓₚᵣₒⱼ(t), which forms the input datum of the encoder. The code C(t) generated by the encoder contains information relating to the stack of images P(t).

FIGS. 5A, 5B and 5C schematically show, at a time t=t₁, a stack of images P(t), a maximum projection image Iₘₐₓₚᵣₒⱼ(t) and a code C(t), respectively.

FIGS. 5D, 5E and 5F schematically show, at a time t=t₂, a stack of images P(t), a maximum projection image Iₘₐₓₚᵣₒⱼ(t) and a code C(t), respectively.

In this example, the code is a data block of 254×254×16 size.

It is not essential to feed the encoder with a maximum projection image. The encoder may be fed with one or a few images from the stack of images. The encoder may also be fed with another transformation of the images of the stack of images, for example an average image or a median image of each image of the stack of images.

Step 120: dimension reduction

In this step, the dimension of each code C(t) is reduced, by being projected into a latent space l of lower dimension than the number of terms forming the code C(t). The dimension of the latent space is preferably less than or equal to 5, preferably less than or equal to 3, and for example equal to 2.

Various methods for reducing dimensions are known. Generally, a method for reducing dimensions or dimensionality allows original coordinates, in an input space, of dimension N, to be represented as coordinates in a latent space of dimension M, with M<N. The dimension of the latent space depends on the dimension of the input space and their complexity. The latent space is passed to from the input space via a projection function f applied to a vector containing all of the terms forming the code.

A plurality of methods for reducing dimensions are known to those skilled in the art. For example, principal component analysis (PCA) is an unsupervised, linear method for reducing dimensionality. Non-linear methods may be implemented, for example the UMAP method (UMAP standing for Uniform Manifold Approximation and Projection).

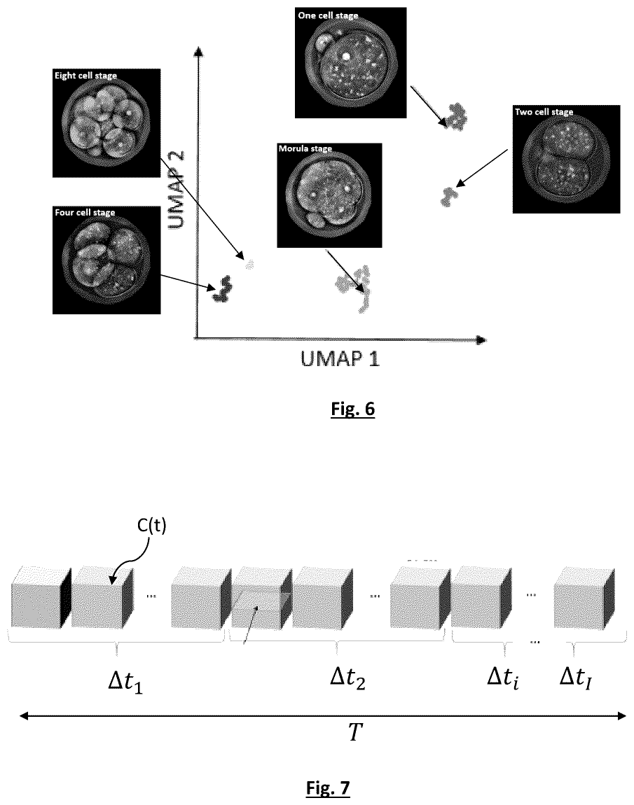

FIG. 6 shows various code projections C(t) corresponding to various respective stacks of images P(t). The latent space is defined in a two-dimensional basis UMAP1, UMAP2. In FIG. 6, each point corresponds to one code C(t), the latter corresponding to one stack of images P(t).

In this example, the dimension-reduction algorithm is applied to compressed information, corresponding to the maximum projection image. The use of the maximum projection image is advantageous when the number of sectional planes is high. However, the dimension-reduction algorithm may be implemented directly on the stack of images, the images of the stack of images forming the input data of the encoder. This is notably the case when the available number of sectional planes is low.

The dimension-reducing step 120 is optional.

### Step 130: Clustering

In this step, a clustering algorithm is implemented, such as to assign a class to each point, in the latent space L, each point corresponding to one stack of images, i.e. to the state of the sample at a time t. A class is thus assigned to the sample, at each given time. The classes may be predefined or not. It is for example possible to implement an unsupervised clustering algorithm, for example a K-means clustering algorithm. Use of other clustering algorithms is conceivable, for example a K-means clustering algorithm, a BIRCH algorithm (BIRCH standing for Balanced Iterative Reducing and Clustering using Hierarchies) or a GMM (GMM standing for Gaussian Mixture Model).

Each class is representative of one state of the sample, at successive times in a time period Δt belonging to the acquisition period T.

FIG. 6 shows 5 clusters, forming 5 classes. During the acquisition period, the sample is successively assigned to each of these classes, depending on its development. Each class corresponds to one time slot Δt, during which the number of cells in the sample remains constant. For example, when the sample is an embryo, five temporal phases of development may be identified, depending on the number of cells contained in the embryo: a first class corresponds to a single cell. A second class corresponds to 2 cells. The third and fourth classes correspond to 4 and 8 cells, respectively. The fifth class corresponds to the formation of a blastocyst. FIG. 6 illustrates the various phases of sample development corresponding to each respective class.

Thus, each class is representative of a number of cells in the sample or of a state of the sample. Assigning a sample to each class thus makes it possible to determine the number of cells N(Δt) in the sample at each acquisition time.

FIG. 7 shows the variation as a function of time in the class of the sample. FIG. 7 shows stacks of images P(t) grouped by class, each class corresponding to one time slot Δtᵢ. i is the index of each time period, ranging from 1 to I, I corresponding to the number of classes.

Preferably, the clustering is unsupervised clustering.

According to one possibility, the clustering is carried out based on each stack of images directly or based on an image obtained by processing each stack of images, and for example based on a maximum projection image, or based on a code resulting from an encoder. The clustering may be carried out by a neural network or another clustering algorithm.

### Step 140: Segmentation—Mask Definition

This step is implemented on each stack of images. In this step, certain images of each stack of images are segmented, in order to define a mask. A mask is a region of a sectional image corresponding to a given object, a cell for example.

In this example, one of the input data of the images is the number of cells N(Δt) to be segmented. This number may be defined based on prior knowledge, or based on measurements taken using another measuring method, in which case steps 110 to 130 are not necessary. When steps 110 to 130 are implemented, the number of cells N(Δt) to be segmented depends on the class assigned to the stack of images.

Another input datum is prior knowledge as to the morphology and/or arrangement of each mask. Thus, the segmentation is carried out by taking into account constraints regarding the geometry of the mask: geometric shape of the outline, absence of “holes” in the mask, maximum degree of overlap between two adjacent masks, or even absence of overlap between two adjacent masks. In the case of cells, the morphological constraints may be:
an outline with a shape that is circular, or ellipsoidal and of eccentricity greater than a threshold value, or within a range of predetermined values;
an area between a minimum area and a maximum area;
a degree of overlap of zero or less than 10% for example.

Generally, a morphological constraint makes it possible to define a morphological correspondence with a mask model, corresponding to a sought biological object.

Other types of constraints may be envisaged, for example constraints relating to physical quantities estimated from each image. It may be a question of a refractive index or phase shift or of a range of refractive indices or phase shifts.

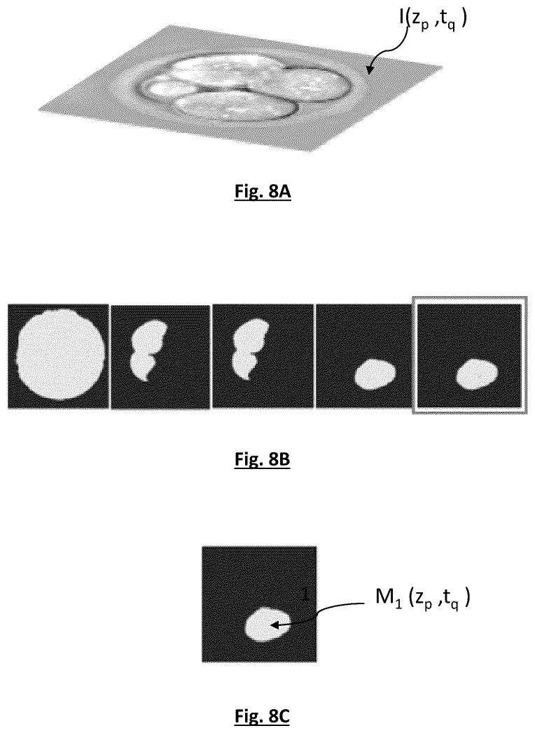

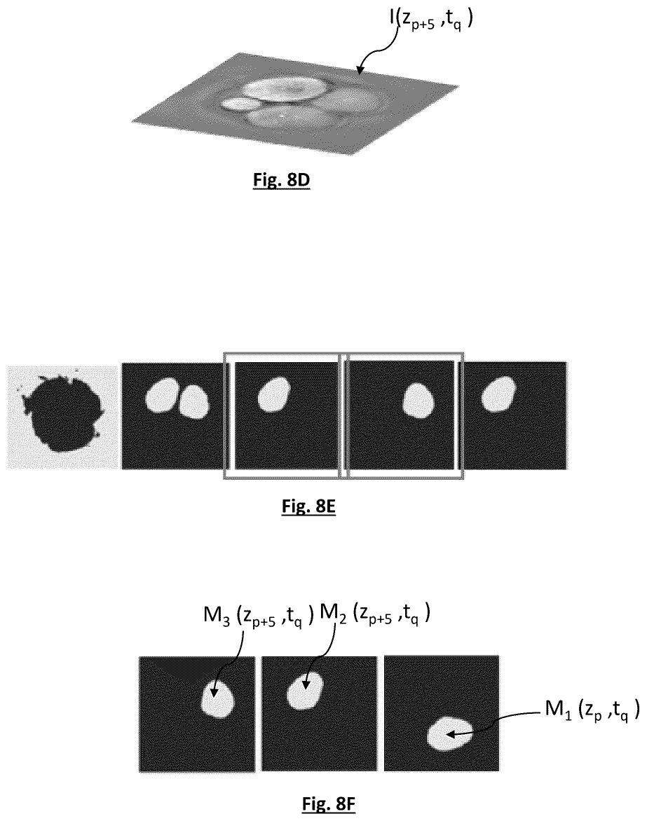

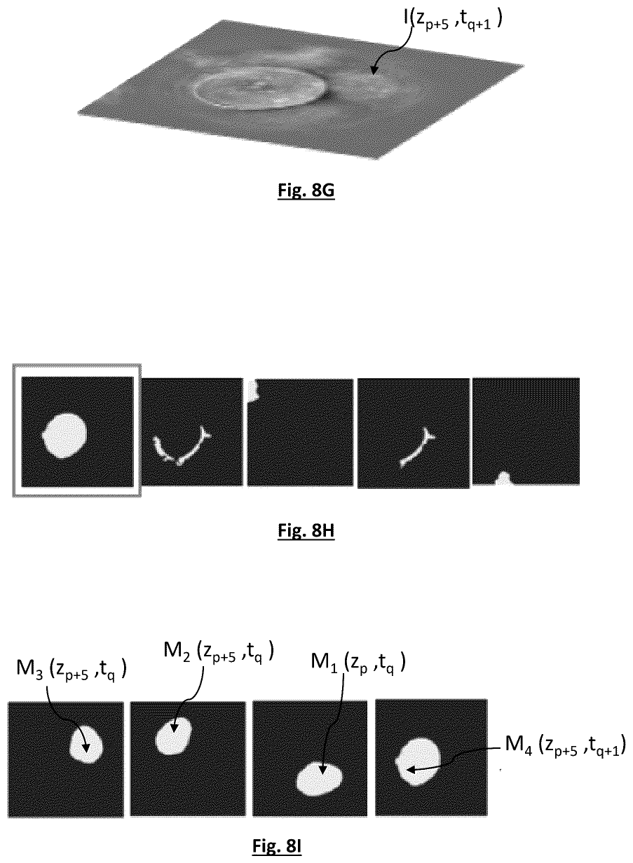

In this step, each image is segmented until a number of masks corresponding to the number of cells N(Δt) in the image is identified. FIGS. 8A to 8I illustrate the process of segmentation and of definition of masks on a stack of images. In this example, a stack of images classified into the class corresponding to a number of cells equal to 4 has been considered. A first image I(zₚ,t_{q}) is taken into account, which image may be chosen arbitrarily. p is a spatial-coordinate index and q is a temporal-coordinate index. It may for example be a question of an image of a median plane of the sample. The image I(zₚ,t_{q}) has been shown in FIG. 8A. The segmentation algorithm makes it possible to define masks, five of which have been shown in FIG. 8B. The segmentation algorithm is for example the Segment Anything Model (SAM) described in J. Cen “Segment Anything in 3D with NerFs”. In general, the number of different masks identified by the algorithm may be greater than 10, or even several tens. FIG. 8B shows only five masks, by way of example. The same goes for FIGS. 8E and 8H, which are described below.

The identified masks are filtered, taking into account the morphological constraints. Only masks meeting the constraints are selected, the other masks being rejected. FIG. 8C shows one mask M₁(zₚ,t_{q}) selected from the masks identified in FIG. 8B. Below, each mask is designated Mᵤ(zₚ,t_{q}), the index u designating the object corresponding to the mask. In this example, u is an integer between 1 and 4 because there are four biological objects (four cells in the sample). Mᵤ(zₚ,t_{q}) is the mask, corresponding to an object u, obtained with the image I(zₚ,t_{q}).

According to one possibility, the filtering involves implementation of an artificial-intelligence algorithm trained using supervised learning, for example a neural network. The neural network may then determine whether a mask resulting from the segmentation corresponds to a biological object likely to be present in the object.

After segmentation and filtering of the first image, a single mask has been selected (namely the mask M₁(zₚ,t_{q}) shown in FIG. 8C). A second image, from the same stack of images, or from a stack of images from the same time slot, is taken into account. The segmentation/filtering process is repeated, such as to identify a mask different from the one selected previously. The new image taken into account belongs to the same time slot Δt. It is spatially and/or temporally offset with respect to the initial image, by one or more spatial and/or temporal increments. FIG. 8D shows the image taken into account: it is the image I(zₚ₊₅,t_{q}). The segmentation algorithm is applied to this image, this allowing masks to be identified, six of which have been shown in FIG. 8E. Filtering makes it possible to select two masks, the latter being framed in FIG. 8E. The selected masks are denoted M₂(zₚ₊₅,t_{q}) and M₃(zₚ₊₅,t_{q}) because they correspond to the second and to the third cell, respectively. Following analysis of the second image I(zₚ₊₅,t_{q}) three masks have been obtained, each being specific to one of three different objects: the mask M₁(zₚ,t_{q}) selected from the first image I(zₚ,t_{q}) and the masks M₂(zₚ₊₅,t_{q}) and M₃(zₚ₊₅,t_{q}) selected from the second image I(zₚ₊₅,t_{q}): these masks have been shown in FIG. 8F.

No mask has been selected for the fourth object. A third image I(zₚ₊₅,t_{q+1}) is taken into account: see FIG. 8G. The third image is segmented, such as to identify masks: see FIG. 8H. From the identified masks, a single mask is selected, which corresponds to the mask framed in FIG. 8H. The selected mask M₄(zₚ₊₅, t_{q+1}) is added to the masks selected during the preceding iterations: see FIG. 8I. For the stack of images, a set of masks corresponding to the number of cells from which the cellular sample is composed is thus obtained.

The step of identifying and selecting the masks is thus an iterative step, comprising the following substeps, shown in FIG. 3B.
taking into account a k^{th} image of the stack of images, or of a stack of images acquired in the same time slot, so that each image taken into account shows the same number of cells in the sample. k designates the rank of the iteration; (substep 141). k is an integer greater than or equal to 1. Preferably, the k^{th} image is chosen from a temporally neighbouring stack of images, i.e. a stack of images temporally offset by a number of temporal and/or spatial increments less than a predetermined threshold with respect to the stack of images in question. The threshold value depends on the spatial separation between two images of a given stack of images, and/or on the time difference between two successively acquired stacks of images. Each (spatial or temporal) threshold is defined on a case-by-case basis.
segmenting the image taken into account, such as to identify masks (substep 142);
filtering the identified masks, taking into account the one or more predefined criteria (substep 143);
reiterating substeps 141 to 143, until the number of masks identified during each iteration reaches the number N(Δt) of cells in the sample (substep 144). More generally, substeps 141 to 143 are reiterated until at least one mask is obtained for each respective cell: either a single mask for each cell, or at least one mask for each cell.

It will be noted that the segmentation is not applied to all the images of the stack of images, but to a sufficient number of images such as to achieve a sufficient number of masks that do not overlap (or that overlap only partially, with a predefined degree of overlap).

The idea is not to obtain one mask for each cell in all the images of all the stacks of images, but a sufficient number of masks, with at least one mask per cell, such as to be able to implement step 150 of prompted segmentation. By sufficient number of masks, what is meant is only 1 mask per cell, or a relatively limited number of masks, for example less than 5 or less than 10 or less than 20 masks per cell. The objective is to achieve an initial definition of the masks for each object, in order to be able to implement the prompted-segmentation algorithm described in connection with step 150, the latter being more efficient.

According to one possibility, the number N(Δt) of biological objects in the sample, in each time slot, is unknown. In this case, substeps 141 to 144 are implemented such as to select masks, corresponding to various objects, until it is no longer possible to define any new masks corresponding to any new objects. It is the segmentation/filtering algorithm that, indirectly, allows the number of biological objects in the sample to be obtained. The number of different biological objects corresponds to the number of respective masks associated with different objects. Each mask is associated with an object based on the position of each mask in an image.

At the end of step 140, in each stack of images, at least one mask is preferably obtained for each biological object. Each mask resulting from this step is associated with one position, which corresponds to the position of the mask in the image in which the mask was defined. Each mask selected in step 140 may be considered to be a base mask, for one cell. The term “base mask” is to be understood to mean that the mask is intended to be used subsequently, to define other masks for said cell, in other images acquired during the same time slot.

The number of base masks defined for a given time slot is limited: it is much lower than the number of images acquired in the time slot multiplied by the number of cells forming the sample. For example, it is less than 2 times or 10 times, or 20 times, or 50 times, or 100 times lower than the number of images acquired in the time slot multiplied by the number of objects.

### Step 150: Propagation—Segmentation of the Entirety of the Stack of Images

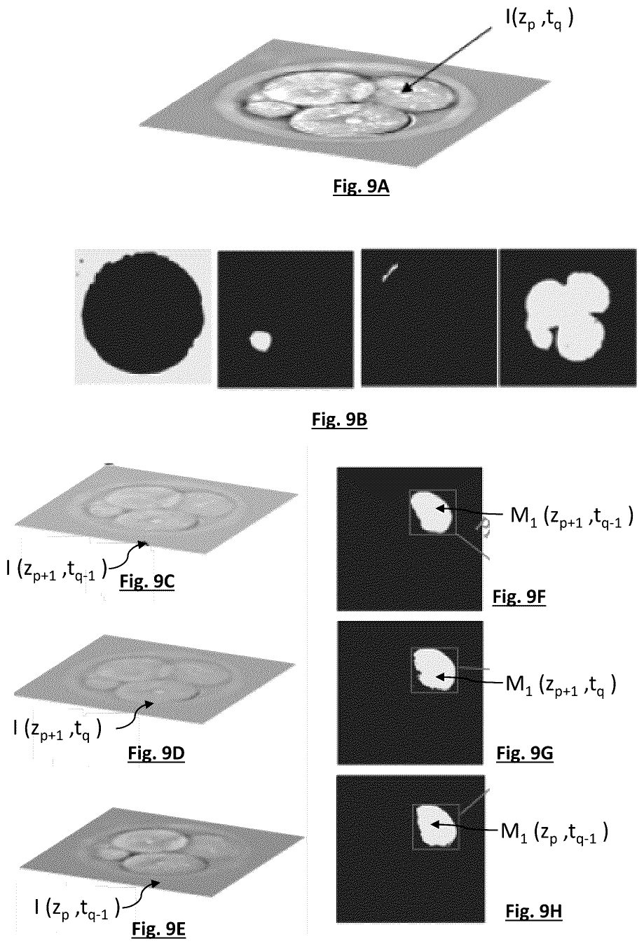

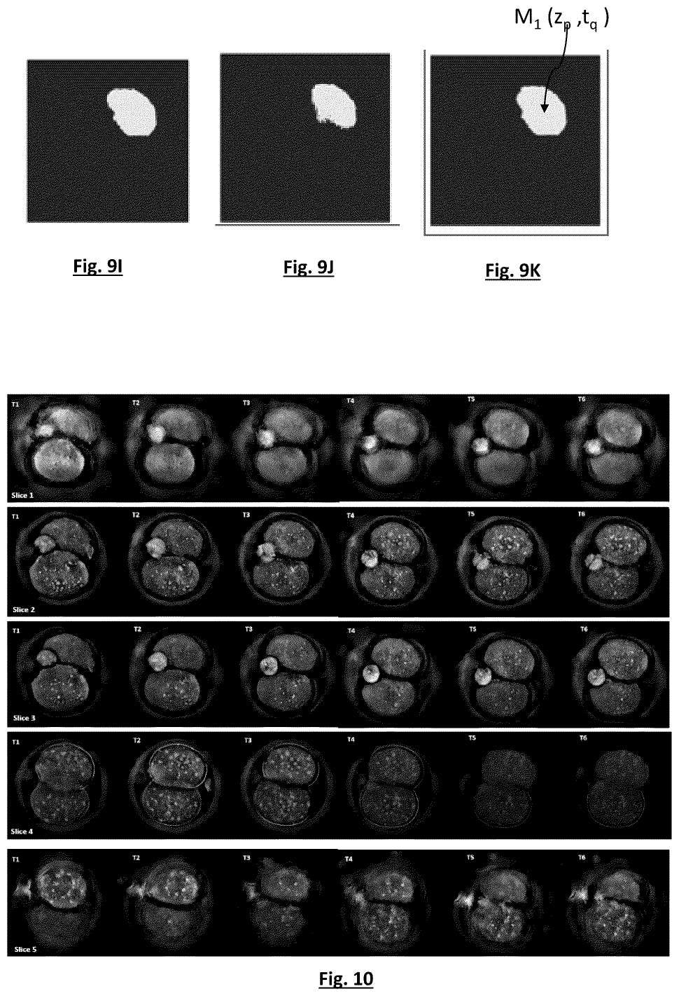

Step 150 is implemented on each stack of images. The masks identified in the images taken into account in step 140 are propagated, from one image to the next, through each given stack of images, such as to obtain a segmentation of each image of the stack of images with a number N(Δt) of masks, N(Δt) corresponding to the number of cells in the sample. Step 150 is illustrated in FIGS. 9A to 9H.

FIG. 9A shows an image I(zₚ, t_{q}) taken from a stack of images.

It would be possible to segment and filter all of the images of each stack of images, the segmentation and filtering being performed independently on each image. However, it has been found that such a solution, which seems obvious, leads to errors. FIG. 9B for example shows an image of a stack of images segmented independently of the other images of the same stack of images. It may be seen that the result is unsatisfactory.

Having observed this, the inventors have suggested a segmentation method, dubbed the prompted (or guided) segmentation method, in which use is made of initial information resulting from a segmentation, such as described in step 140, carried out:
in an image offset temporally by one or more temporal increments, the number of temporal increments being less than a certain threshold so that the offset image may be considered close enough “temporally” to the processed image;
and/or in an image offset spatially by one or more spatial increments, the number of spatial increments being less than a certain threshold so that the offset image may be considered close enough “spatially” to the processed image.

The underlying idea is to perform, on the images of a stack of images, prompted segmentation guided by the position of segmentation masks identified in images acquired during the same time slot Δt, and that are preferably temporally or spatially neighbouring. The quality of the masks generated by the prompted segmentation algorithm is far superior to the quality of masks generated by the segmentation algorithm without prompting (i.e. without guidance).

For each of the cells from which the cellular sample is composed, the segmentation of the images of a stack of images is carried out gradually, starting with reference images. By reference image, what is meant is an image of the stack of images in which the relevant cell has been segmented.

Step 150 comprises the following substeps:
Substep 151: selecting a stack of images belonging to a time slot Δt in which the sample contains N(Δt) cells.
Substep 152: selecting one cell from the N(Δt) cells that the sample contains.
Substep 153: selecting a reference image, in which the mask of the cell has been identified. The reference image may be an image of the stack of images, or an image of a stack of images temporally offset from the stack of images in question, and belonging to the same time slot Δt. Preferably, the reference image neighbours the stack of images temporally, being distant by 5 temporal increments, for example, from the stack of images selected in substep 151.
Substep 154: selecting an image to be analysed in the stack of images in question. Preferably, the analysed image is formed in a sectional plane neighbouring the sectional plane in which the reference image is formed. By neighbouring sectional plane, what is meant is a sectional plane the coordinate of which, along the Z-axis, is offset by one or a few spatial increments. The spatial offset is for example less than 5 spatial increments.
Substep 155: based on the position of the mask in the reference image, segmenting the image selected in substep 154. One way of determining the position of the mask in the reference image is to define a bounding box in the reference image. The bounding box frames the mask. The bounding box defined in the reference image is transferred to the analysed image. The segmentation algorithm takes into account the position of the bounding box in the analysed image to identify a mask. Thus, the segmentation algorithm is guided, or “prompted” by the mask previously defined in the reference image.

This requires the use of a prompted segmentation algorithm, segmentation in an image being performed based on an indication as to the probable position of the mask: said position may be defined by a bounding box, by the centre of the mask or by a point cloud.

Substep 155 may be implemented successively taking into account a plurality of reference images, in which the same cell has been demarcated by various respective segmentation masks. Each reference image is preferably located in a spatial or temporal neighbourhood of the analysed image. In this case, the mask, corresponding to the cell selected in substep 152, is transferred, from each reference image, to the analysed image. As many bounding boxes are transferred as there are reference images to be taken into account. The segmentation algorithm makes it possible to determine as many segmentation masks as there are transferred bounding boxes. A filtering step makes it possible to select, from among the various segmentation masks, the most suitable segmentation mask. The filtering is carried out as described in connection with step 140.

After substep 155, the analysed image becomes a reference image for the cell selected in substep 152. It may be used, in turn, to guide segmentation of another image of the cell.
Substep 156: selecting another cell. Substeps 153 to 155 are reiterated for each of the N(Δt) cells contained in the sample during the time slot Δt.
Substep 157: selecting another stack of images in the same time slot Δt. After all the images of a given stack of images have been segmented, for all the N(Δt) cells, substeps 153 to 156 are reiterated for another stack of images.

Step 150 may be implemented for one or more time slots Δt. Step 150 may be implemented for all or some of the N(Δt) cells in each time slot.

FIGS. 9C, 9D and 9E respectively show, for a first cell:
a reference image I(zₚ₊₁,t_{q-1}), belonging to a stack of images P(t_{q-1});
a reference image I(zₚ₊₁,t_{q}), belonging to a stack of images P(t_{q});
a reference image I(zₚ,t_{q-1}), belonging to a stack of images P(t_{q-1}).

The images shown in FIGS. 9C, 9D and 9E are used as reference images to segment an image I(zₚ,tₐ) in order to identify a segmentation mask corresponding to the first cell. FIGS. 9F, 9G and 9H show the segmentation masks M₁(zₚ₊₁,t_{q-1}), M₁(zₚ₊₁,t_{q}) and M₁(zₚ,t_{q-1}) selected during step 140, based on the images I(zₚ₊₁,t_{q-1}), I(zₚ₊₁,t_{q}) and I(zₚ,t_{q-1}), respectively. These masks form base masks for the first cell. A bounding box has also been shown around each mask.

Each bounding box was then transferred to the image I(zₚ,t_{q}). The segmentation masks shown in FIGS. 9I, 9J and 9K were obtained as a result, based on transfer, to the image I(z, t), of the bounding boxes corresponding to the masks M₁(zₚ₊₁,t_{q-1}), M₁(zₚ₊₁,t_{q}) and M₁(zₚ,t_{q-1}), respectively. After filtering, the retained mask is the one shown in FIG. 9K. The latter is designated M₁(z_{q}, t_{q}) because it corresponds to the mask defined in the image I(zₚ, t_{q}) for the first cell (u=1). This mask may be used to prompt a segmentation of an image of the stack of images P(t_{q}) or an image, preferably neighbouring, of another stack of images of the same time slot Δt.

Following step 150, for each stack of images P(t), and for each cell, a mask, called the 3D mask, which corresponds to all the masks defined, for said cell, in each image I(z, t) of the stack of images, is obtained.

### Step 160: Observation

In this step, the masks defined in each image, for each cell, are transferred to the images of the stack of images, in order to highlight each cell. For example, each cell may be outlined or coloured with a certain colour, to allow it to be seen better. For example, each cell may be coloured with a different colour, such as to discriminate between the cells.

Moreover, obtaining 3D masks for each cell makes it possible to access information on morphological changes undergone by each cell during the development of the cellular sample. It is for example a question of quantifying changes in the area or shape of each 3D mask.

FIG. 10 shows, for a mouse embryo containing 4 cells, an application of the 3D segmentation. FIG. 10 shows various images of sectional planes. Each row of FIG. 10 corresponds to the same sectional plane and each column corresponds to one time.

Although described in connection with an example where the cellular sample was an embryo, notably a non-human embryo, the invention is applicable to 3D segmentation of other types of biological samples, for example organoids, multicellular organisms, or other 3D biological structures, comprising biological objects the number and/or shape of which change over time. CLAIMS

1. A segmenting method, for carrying out 3D segmentation of a sample, the sample comprising at least one biological object, the sample developing over time, such that at least one biological object divides or changes shape or position over time, the method comprising:
a) at various times, acquiring a stack of images of the sample, each image of the stack of images showing one sectional plane of the sample, each stack of images forming a three-dimensional representation of the sample, in various planes, at a given time, the times defining time slots, such that during each time slot the sample contains the same number of biological objects, at least one stack of images being acquired in each time slot;
b) segmenting at least one image of a stack of images such as to define masks in said image, each mask being bounded by a closed outline;
c) selecting masks defined in step b) depending on predefined selection criteria, such that each selected mask corresponds to one biological object, each selected mask being associated with a position corresponding to a position of the mask in the segmented image;
d) reiterating steps b) and c) on another image of a stack of images of the same time slot, until at least one selected mask is obtained for each biological object of the sample;
e) selecting a stack of images, acquired during a time slot of interest;
f) for at least one biological object, and using at least one mask, selected in the time slot of interest and corresponding to the biological object, carrying out segmentation of each image of the stack of images selected in step e), such as to determine, in a plurality of images of said stack of images, masks corresponding to the biological object, the segmentation being guided by the position of the selected mask;
g) repeating steps e) to f) for various stacks of images in at least one time slot;
steps b) to g) being implemented by a processing unit.

2. The method according to claim 1, wherein steps e) and f) are repeated for each stack of images of each time slot.

3. The method according to claim 1, wherein step f) is repeated for each biological object of the sample, in at least one time slot.

4. The method according to claim 1, wherein the method comprises, prior to step b), obtaining a number N(Δt) of biological objects contained in the sample, during each time slot, N(Δt) being an integer greater than or equal to 1.

5. The method according to claim 4, comprising, prior to step b), determining the number of biological objects, in at least one time slot, from at least one stack of images acquired during the time slot.

6. The method according to claim 5, wherein:
the acquisition times are distributed between various time slots, with each time slot corresponding to a number of biological objects in the sample;
the method comprises, prior to step b), clustering each stack of images, such as to assign each stack of images to one of said time slots.

7. The method according to claim 6, comprising prior to step b):
i) applying a dimension-reduction algorithm to each stack of images, such as to assign a coordinate to each stack of images in a latent space;
ii) in the latent space, assigning each coordinate to a class;
iii) determining the number of biological objects in the sample, for each stack of images, depending on the class corresponding to the stack of images.

8. The method according to claim 7, wherein step i) comprises using an encoder neural network to obtain, from each stack of images, a code corresponding to each stack of images, the dimension-reduction algorithm being applied to the code.

9. The method according to claim 7, wherein step i) comprises forming an image representative of each stack of images, to be fed to the encoder neural network.

10. The method according to claim 9, wherein the image representative of each stack of images is a maximum projection image established for each stack of images.

11. The method according to claim 1, wherein, in step c), at least one selection criterion is chosen from:
a morphological criterion of the mask;
a maximum overlap between two selected masks.

12. The method according to claim 1, wherein step c) comprises determining a physical property of the sample in a portion of the image corresponding to each mask, the selection criterion depending on the value of said physical property.

13. The method according to claim 12, wherein the physical property is an optical property of the sample.

14. The method according to claim 1, wherein:
in step a), two successively acquired stacks of images are offset by one temporal increment, two adjacent images of a given stack of images being offset by one spatial increment;
each image in which at least one mask has been selected in a step c) is a reference image for the biological object corresponding to the mask;
step f) comprises:
f-i) selecting a biological object;
f-ii) selecting an image in which no mask has been defined for the selected biological object, the selected image having a spatial coordinate and a temporal coordinate;
f-iii) for the biological object selected in substep f-i), selecting a mask corresponding to the selected biological object in a neighbouring reference image, neighbouring the image selected in substep f-ii), the neighbouring reference image having respective spatial and temporal coordinates offset by a respective number of spatial and temporal increments less than a predetermined threshold;
f-iv) transferring the position of the mask selected in substep f-iii) to the image selected in substep f-ii);
f-v) carrying out segmentation of the image selected in substep f-ii), using the position transferred in substep f-iv), such as to define a mask for the biological object selected in substep f-i), the segmentation being guided by said position of the mask.

15. The method according to claim 14, wherein following step f-v), the image selected in substep f-ii) becomes a reference image for the biological object selected in substep f-i).

16. The method according to claim 14, wherein
in substep f-iii), the position of the selected mask is defined by a bounding box framing said mask;
substep f-iv) comprises transferring the bounding box to the image selected in substep f-ii).

17. The method according to claim 14, wherein substeps f-i) to f-v) are carried out such as to obtain one mask for each biological object of the sample in each image of each stack of images.

18. The method according to claim 1, wherein the sample is a multicellular organism, each biological object being one cell.

19. A device for observing a sample, comprising:
an acquisition system (1), configured to form a stack of images of the sample at various times, each stack of images showing the sample at a given time, the times defining time slots, during which the sample contains the same number of biological objects, such that at least one stack of images corresponds to each time slot;
a processing unit, configured to implement steps b) to g) of a method according to claim 1, based on the respective stacks of images formed at the various times.

20. A medium that is connectable to a computer and that contains instructions for implementing steps b) to g) of the method according to claim 1 based on stacks of images of a sample.
